(19) United States
(12) Patent Application Publication
(10) Pub. No.: US 2015/0118293 A1
(43) Pub. Date: Apr. 30, 2015

(54) NOVEL DELIVERY SYSTEM FOR ANTICANCER DRUG BASED ON SHORT-CHAIN- LENGTH POLYHYDROXYALKANOATE NANOPARTICLES

(71) Applicants: Farha Masood, Islamabad (PK); P. Chen, Waterloo (CA); Tariq Yasin, Islamabad (PK); Abdul Hameed, Islamabad (PK)

(72) Inventors: Farha Masood, Islamabad (PK); P. Chen, Waterloo (CA); Tariq Yasin, Islamabad (PK); Abdul Hameed, Islamabad (PK)

(21) Appl. No.: 14/066,899

(22) Filed: Oct. 30, 2013

(51) Int. Cl. A61K 47/34; A61K 31/437; A61K 9/14; A61K 9/48

(52) U.S. Cl. CPC A61K 47/34; A61K 9/48; A61K 31/437; A61K 9/14
USPC 424/451; 514/285

(57) ABSTRACT

The invention relates to the fabrication of novel and improved delivery system comprising of short-chain-length polyhydroxyalkanoates (scl-PHA) nanoparticles having anticancer drug ellipticine (EPT) encapsulated therein for oral administration. The scl-PHA is synthesized by indigenous bacterial isolate Bacillus cereus FC11 (NCBI GenBank accession number JN593010) using modified GTYN medium.

### FIELD OF THE INVENTION

The present invention relates to the improved delivery system comprising of scl-PHA nanoparticles having anticancer drug EPT encapsulated therein for oral administration wherein scl-PHA is synthesized by Bacillus cereus FC11.

### BACKGROUND OF INVENTION

Nanoparticulate systems are made up of sub-microscopic colloidal particles having a diameter of preferably 1-1000 nm (1,000 nm=1 μm). The nanoparticles can be formed from both the natural and synthetic polymers and are used as carriers for therapeutics, vaccines, and genes.

The main advantage of drug delivery systems based on polymeric nanoparticles is a conversion of the poorly soluble and highly toxic therapeutic into a pharmacologically active dose by increasing its efficacy and bioavailability with minimum drug dosing frequency over a longer period of time while evading toxicity in the normal cells.

The use of biodegradable polymers is well established for the fabrication of drug delivery systems. For instance, considerable research has been devoted to encapsulating therapeutic agents into polyesters such as poly (lactic acid) (PLA), poly (lactic acid-co-glycolic acid) (PLGA), poly (ε-caprolactone-co-glycolic acid) and polyhydroxyalkanoic acid (PHA).

The release of encapsulated therapeutics from the polymer based nanoparticles after administration to human body is reported to occur as the polymer degrades and/or as water diffuses into the polymer to leach out the therapeutic at a controlled rate. European Patent 2150237A1 discloses biocompatible polymeric nanoparticles for drug delivery system comprising of poloxamer, polyethylene glycol (PEG) and paclitaxel (PTX). European Patent 0467389B1 describes a drug delivery system comprising of calcitonin and a hydrophobic biodegradable PLGA. European Patent 2319503A2 disclosure relates to a drug delivery carrier including a biocompatible chitosan and a hydrophobic group conjugated to the polymer.

PHAs are the natural polyesters comprising of the repeating hydroxy acids monomeric units. The microorganisms belonging to 90 different genera are reported to accumulate PHAs from renewable resources as an intracellular energy and carbon storage compounds under the nutrient stress conditions [Jendrossek, (2009) J Bacteriol 191(10): 3195-3202]. PHAs are biodegradable, biocompatible and non-toxic in nature. Moreover, the D-(−)-3-hydroxybutyrate (3-HB) produced as a result of PHA degradation is a normal constituent of human blood plasma as well as non-toxic to the surrounding tissues [Tokiwa and Calabia, (2004) Biotechnol Lett 26; 1181-1189].

Short-chain-length PHAs (scl-PHAs) are made up of 3-hydroxy monomers with chain lengths of C4 and C5, while medium-chain-length PHAs (mcl-PHAs) are composed of 3-hydroxy monomers with chain lengths from C6 to CI4. Approximately 150 different monomeric units of PHAs are identified as homopolymers or copolymers [Steinbüchel and Lütke-Eversloh, (2003) Biochem Eng J 16: 81-96]. WO2012071657 A1 discloses the monomeric composition of PHAs depends both on the type bacterial strain(s) used and on the type of carbon source(s) supplied. The particular selected microbial strain will typically produce via different metabolic pathways either scl-PHA or mcl-PHA but not the both.

In 2007, the FDA approved the practical applications of PHA in biomedical areas. U.S. Patent Publication 2012/0328523 A1 discloses that PHAs are suitable for use in in vivo applications such as in tissue coatings, stents, sutures, tubing, bone, prostheses, bone or tissue cements, tissue regeneration devices, wound dressings and drug delivery.

The homopolymer and copolymers of PHA i.e., poly-3-hydroxybutyrate (PHB), poly-3-hydroxybutyrate-co-3-hydroxyvalerate (PHBV), poly-3-hydroxybutyrate-co-3-hydroxyhexanoates (PHBHHx), poly-3-hydroxybutyrate-co-4-hydroxybutyrate-co-3-hydroxyhexanoates (P3HB-co-4HB-co-3HHx), poly-3-hydroxybutyrate-co-6-hydroxyhexanoates-co-3-hydroxyhexanoates (P3HB-co-6HHx-co-3HHx) and poly-3-hydroxybutyrate-co-poly-3-hydroxyoctanoate (PHB-co-PHO) are used as drug carrier [Lu et al., (2011) Appl. Microbiol. Biotechnol. 89:1423-1433; Shi et al. (2010) Nano Lett. 10: 3223-3230; Lu et al. (2010) J. Appl. Polym. Sci. 116: 2944-2950; Xiong et al. (2010) J. Biomater. Sci. 21:127-140; Francis et al. (2011) Int. J. Mol. Sci. 12:4294-314; Sun et al. (2007) Biomaterials 28:3896-903; Bayram et al. (2008) J Bioactive Compatible Polym 23: 334; Zhang et al. (2010) Eur J Pharm Biopharm 6; 10-16; Shah et al. (2010) Int J Pharm 400; 165-175]. For instance, U.S. Patent Publication 2012/0328523 A1 and European Patent 1163019 A1 disclose the PHA compositions provide favorable mechanical properties, biocompatibility, and degradation times within desirable time frames under physiological conditions for biomedical applications. The fabrication of the novel homo-polymer, co-polymer and modification of their side chains are key factors in controlling the rate of drug release from polymer based delivery systems [Langer R, Folkman J. Nature 1976; 263(5580):797-800: Langer, R., Nature (1998) 392, 5].

In nature, both the enzymatic and hydrolytic degradation of PHAs is dependent upon the surface area, temperature, pH, molecular weight and crystallinity. T\ddot{υ}resin et al. (2001) J. Biomater. Sci. Polymer Edn. 12:195-207 have described the use of poly-3-hydroxybutyrate-co-4-hydroxybutyrate (P3HB4HB) to develop controlled release systems for the treatment of osteomyelitis. European Patent 1651273 A1 disclosure relates to poly-4-hydroxybutyrate (P4HB) matrices for sustained drug delivery since it is slowly degraded and cleared by the patient's body after use. Regardless of this encouraging progress there still exists a need for fabrication and designing of new and improved drug delivery systems. It would also be desirable to design innovative systems that can be loaded with high amounts of sparingly soluble drugs to provide prolonged release for treatments of various diseases.

The natural plant alkaloid ellipticine (5,11-dimethyl-6H-pyrido[4,3-b]carbazole) (EPT) is isolated from the leaves of the evergreen tree Ochrosia elliptica. The EPT is a potent mutagen, anti-HIV and antitumor agent [Stiborová et al. (2001) Biochem Pharmacol 62:675-684]. EPT showed the significant anticancer activity against the brain tumor cell line and non-small-cell-lung-cancer. But, the application of EPT in clinical trials is limited due its poor water solubility which is approximately 0.62 mM at neutral pH [Wu et al. (2012) Int J Nanomedicine 7:3221-33; Wu et al. (2011) Mol Biosyst 7; 2040-2047; Stiborová et al. (2001) Biochem Pharmacol 62:675-684].

### BRIEF SUMMARY OF THE INVENTION

Taking into account the problems of the prior art described above, the object of the present invention is to develop improved delivery system comprising of scl-PHA nanoparticles having EPT encapsulated therein.

Another object of this invention is to propose an oral drug delivery system of scl-PHA nanoparticles having an EPT encapsulated therein which has enhanced the bioavailability of drug.

A further object of this invention is to propose a drug delivery system of scl-PHA nanoparticles having an EPT encapsulated therein which is effective against lung cancer. It is another object of the present invention to use this scl-PHA nanoparticle based drug delivery system to increase the bioavailability of other hydrophobic therapeutics for cancer therapy.

The improved drug delivery system of the present invention comprising of scl-PHA nanoparticles having EPT encapsulated therein show desired particle sizes and uniform particle size distribution and are suitable for use in drug delivery. The biodegradable, biocompatible and non-toxic scl-PHA nanoparticles of the present invention are thus safe for use in the body. Further, after being administered in the body, the scl-PHA nanoparticles of the present invention, with a high content of sparingly soluble anticancer therapeutic entrapped therein, can safely deliver the drug to target sites and can stably release the drug at a controlled rate.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1 depicts a HPLC chromatogram showing the same retention time of crotonic aid (by-product) obtained by digestion of PHA in the presence of concentrated H₂SO₄; upper one is obtained from the conversion of PHBV-S and the bottom one is obtained from the conversion of PHA sample produced by Bacillus cereus FC11 strain.

FIG. 2 depicts a graph showing the results of Fourier Transform Infrared spectra of PHBV-S and polymer obtained from Bacillus cereus FC11 strain IR (cm⁻¹): γ 1375 (CH, v, scissoring and bending), 1456 (CH, v), 2932 (CH, s), 2975 (CH, s), 1720 (C═O, s, br), 1275 (C—O, s).

FIG. 3 depicts the ¹H-NMR spectrum of the copolymer PHBV obtained from Bacillus cereus FC11 strain (CdCI₃, ppm); δ_{H} 0.87 (7H, CH₃), 1.24 (5H, CH₃), 1.62 (6H; CH₂), 2.62 (1H, CH₂; 3H, CH₂), 5.24 (2H, CH₂; 4H, CH).

FIG. 4 depicts the spectrum of ¹³C-NMR of PHBV obtained from Bacillus cereus FC11 strain (CdCI₃, ppm); δ_{C} 169.12 (1C, 4C, C═O), 67.60 (3C, CH; 6C, CH), 40.78 (5C, CH₂), 30.01 (2C, CH₂), 23.58 (8C, CH₂), 19.75 (7C, CH₃), 9.39 (9C, CH₃).

FIG. 5 depicts a graph showing the size distributions of blank and drug loaded scl-PHA nanoparticles. The bars represent the average +/−SD.

FIGS. 6A and 6B depict scanning electron micrographs of blank and drug loaded scl-PHA nanoparticles.

### DETAILED DESCRIPTION OF THE INVENTION

A list of definitions of terms used in the description of the present invention is described below:

The term “microbes” generally refers to, for example, microorganisms such as bacteria, fungi, viruses, like biological entitles and combinations thereof. The terms microbes and microorganisms will be used interchangeably herein.

The term “natural resource” refers to a material that occurs in the natural environment, and may comprise one or more biological entities.

The term “polyester” refers to the category of polymers that contains a functional ester group as their main chain.

The term “short-chain-length polyhydroxyalkanoates”, also referred to herein as “scl-PHAs” are poly(3-hydroxyalkanoates) classified as having 3-5 carbons in their repeating units.

The term “polyhydroxy acids” as used herein means a polymer of repeating hydroxyl acid monomer units. The term “polyhydroxalkanoates”, also referred to herein as “PHAs”, are renewable, thermoplastic, linear, aliphatic polyesters and/or co-polyesters produced by bacterial fermentation of sugars and/or lipids. PHAs are generally formed within bacterial cells as refractive granules comprising: (i) homopolyesters with the same hydroxyalkanoic acids, or (ii) copolyesters with different hydroxyalkanoic acids.

The term “C3” is used herein for convenience to represent a polyhydroxyalkanoate monomer that has three carbons (three of the carbons in its backbone) and is named polyhydroxypropionate.

The term “C4” is used herein for convenience to represent a polyhydroxyalkanoate monomer that has four carbons (three of the carbons in its backbone and one carbon in its side chain) and is named polyhydroxybutyrate.

The term “C5” is used herein for convenience to represent a polyhydroxyalkanoate monomer that has five carbons (three of the carbons in its backbone and two carbons in its side chain) and is named polyhydroxypentanoate.

The term “nanoparticle” is to be interpreted broadly to refer to a particle which has a dimension in the nano-size range, or less than about 1000 nm, particularly less than about 250 nm, or more particularly between about 100 nm to about 200 nm. Where the nanoparticle is not a spherical particle, the above dimension may refer to the dimension of an equivalent spherical particle. Hence, the dimension may refer to the diameter of the nanoparticle (or equivalent spherical particle thereof).

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means+/−5% of the stated value, more typically +/−4% of the stated value, more typically +/−3% of the stated value, more typically, +/−2% of the stated value, even more typically +/−1% of the stated value, and even more typically +/−0.5% of the stated value. Throughout this disclosure., certain embodiments may be disclosed in a range format. It should be understood that the description in range format is merely for convenience and brevity and should not be construed as an inflexible limitation on-the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Biodegradable means that the polymer must break down or dissolve away in vivo, preferably in less than two years, and more preferably in less than one year. Biodegradation refers to a process in an animal or human. The polymer may break down by surface erosion, bulk erosion, hydrolysis or a combination of these mechanisms.

Biocompatible refers to materials that are not toxic, and do not elicit severe inflammatory or chronic responses in vivo. Any metabolites of these materials should also be biocompatible. The biocompatible polymer used in the present disclosure needs not have a particularly limited molecular weight. Specifically, it may have an average molecular weight 1,000 kDa or smaller, more specifically 300 kDa or smaller, most specifically 100 kDa or smaller. The polymeric material may be selected considering its characteristics, the properties of the corresponding synthetic drug, or the like.

The drug delivery carrier according to the present disclosure may be prepared by directly encapsulating the hydrophobic drug within the polymer and/or attached to the surface of the polymeric material having a carboxyl group via hydrophobic interaction.

In addition to the improved adsorption between the polymeric material and the drug, the sustained release of the drug adsorbed to the drug delivery carrier is attained. This is because the polymeric material is decomposed very slowly in the body. As the biocompatible polymer is decomposed by enzymes, the drug adsorbed to the polymer chain is released slowly over time in a sustained manner.

Since there are various biocompatible natural or synthetic polymers and various hydrophobic therapeutics that can be used to prepare the drug delivery carrier according to the present disclosure and the drug delivery carriers resulting from various combinations thereof have varying physical and chemical properties, a desired drug delivery carrier may be prepared depending on purposes.

In greater detail, the drug delivery system comprises of nanoparticles of scl-PHA represented by the following Chemical Formula 1, having anticancer drug EPT represented by the following Chemical Formula 2, encapsulated therein:

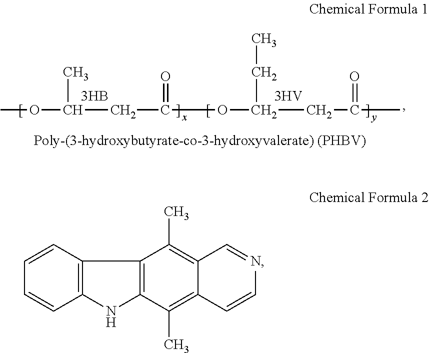

In the description of the present invention, the following abbreviations may be used: short chain length, scl; polyhydroxyalkanoates, PHA; ellipticine, EPT; poly-3-hydroxybutyrate, PHB; poly-3-hydroxybutyrate-co-3-hydroxyvalerate, PHBV; poly-3-hydroxybutyrate-co-3-hydroxyhexanoates, PHBHHx; poly-3-hydroxybutyrate-co-4-hydroxybutyrate-co-3-hydroxyhexanoates, P3HB-co-4HB-co-3HHx; poly-3-hydroxybutyrate-co-6-hydroxyhexanoates-co-3-hydroxyhexanoates, P3HB-co-6HHx-co-3HHx; polyvinyl alcohol, PVA; 3-hydroxybutyrate, 3-HB; 3-hydroxyvalerate, 3-HV; medium-chain-length, mcl; poly-3-hydroxybutyrate-co-4-hydroxybutyrate, P3HB-co-4HB.

A better understanding of the present invention may be obtained through the following examples which are set forth to illustrate, but are not to be construed as the limit of the present invention.

### Example 1

### Characterization of PHBV Copolymer

The sample containing 0.01 to 500 mg PHA material obtained by fermentation from Bacillus cereus FC11 was digested in 1 mL of concentrated sulfuric acid at 100° C. for 30 min in water bath. The test tubes were then cooled on ice and diluted with 4 mL volume of 0.014 N H₂SO₄ with rapid mixing. The injection volume was 10 μL and sample concentrations were ranging from 0.2-560 μg/mL. The samples was eluted from an IC-Pak™ Ion-Exclusion 50 A° 7 μM (300×7.8 mm) preceded by an ion-exclusion guard column of IC-Pak™ Ion-Exclusion using 0.014 N H₂SO₄ at 0.70 mL/min flow rate and 60° C. The amount of crotonic acid produced from PHA was calculated from the regression equation derived from known standard PHBV (Sigma), which here after called as PHBV-S. The structural analysis was performed using FTIR spectrophotometer (Nicolet model 6700; Thermo Electron Corp, Marietta, Ohio) and compared with standard PHBV (S). The NMR analysis was done at a room temperature using an AVANCE 300 B spectrometer (Bruker Daltonics, Billerica, Mass.). About 20 mg of polymer sample was dissolved in chloroform-d (CDCl₃) and 0.75 mL of solution was placed in NMR tubes. The ¹H and ¹³C-NMR spectra were recorded at room temperature.

### Results

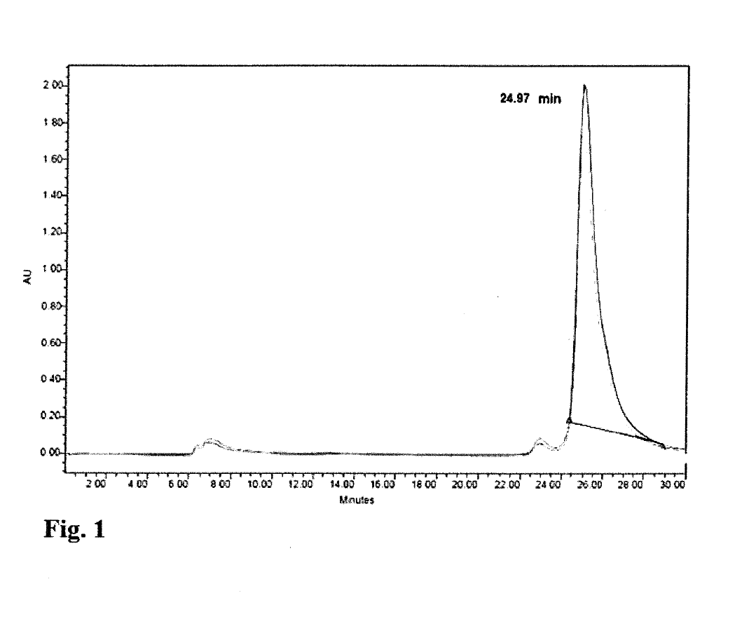

PHBV-S was first converted into crotonic acid, a by-product obtained after digestion of PHBV-S in the presence of concentrated H₂SO₄. A peak of crotonic acid was eluted at around 24.97 min and second peak at 7.5 min was due to the solvent elution containing digested cell residue. The PHA obtained from B. cereus FC11 was also converted into crotonic acid and it was also eluted at 24.97 min. The appearance of the single peak at same retention time was indicating that the PHA produced in our case is same (FIG. 1).

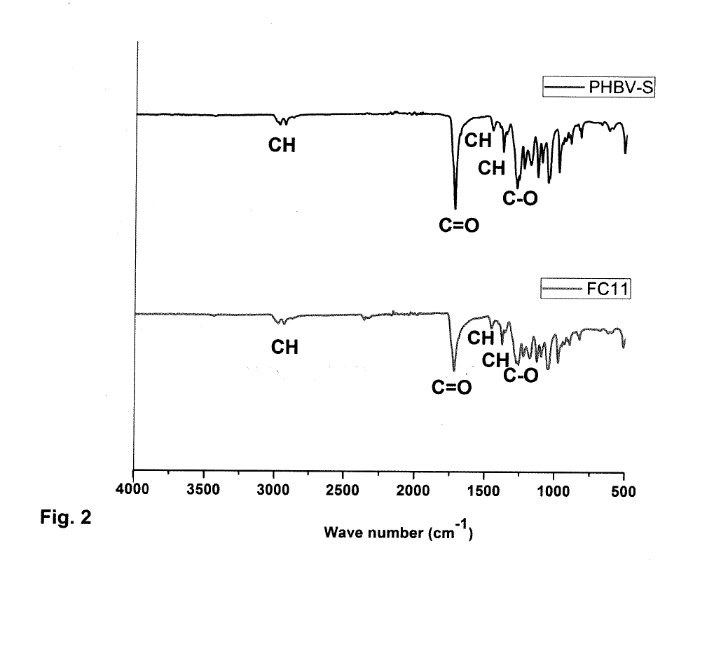

FTIR spectrum of PHBV-S showed the absorption peaks at 1720 and 1275 cm⁻¹, which were representing the C═O and C—O stretching. The absorption peaks appeared at 1375, 1456, 2932 and 2975 cm⁻¹ were attributed to C—H vibration form —CH₂, —CH₃ and —CH bonds. FTIR spectra of purified PHA obtained from B. cereus FC11 showed absorption bands at 1275, 1375, 1456, 1720, 2932 and 2975 cm⁻¹, which were similar to peaks appeared in spectrum of PHBV-S (FIG. 2).

According to Bloembergen et al. (1986) Macromolecules 19; 2865-2871 and Liu et al. (2010) J Appl Polym Sci 116(6); 3225-3231 the characteristic peaks at 0.90 and 1.25 ppm in the ¹H-NMR spectrum can be used to determine the HV composition of PHBV according to the following equation:

$$HV composition ( \% ) = \frac{Area {CH}_{3} ( HV )}{Area {CH}_{3} ( HV ) + Area {CH}_{3} ( HB )} \times 100$$

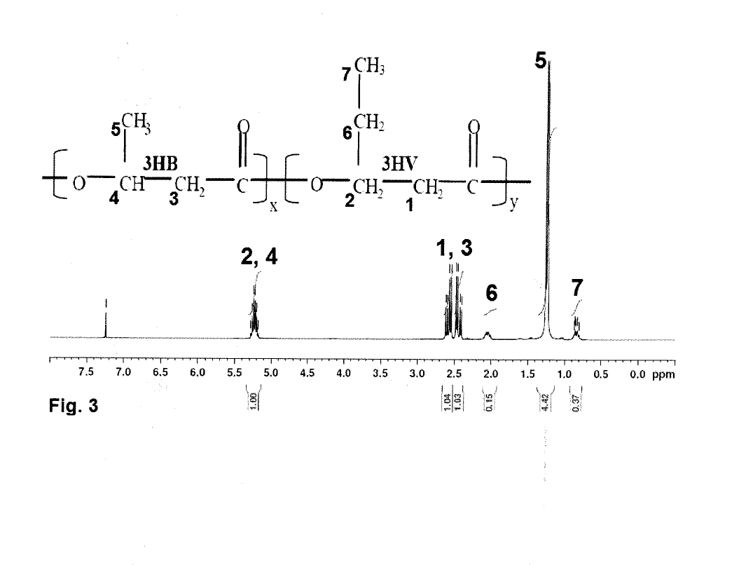

The integration of the area under the peaks at 0.87 (7) and 1.24 (5) ppm was done and it was found that polymer produced by B. cereus FC11 contained two monomeric units: 92 mol % of 3-HB and 8 mol % of 3-HV, hereafter referred as PHBV-8 (FIG. 3). Furthermore, the characteristic peaks at 0.87 (7) and 1.24 (5) ppm were corresponding to —CH₃ groups of hydroxybutyrate (HB) and hydroxyvalerate (HV). The two peaks appeared at 2.62 (1, 3) ppm were attributed to the —CH₂ groups of HB and HV. The peak obtained at 1.62 (6) ppm was corresponding to —CH₂ group of HV. While, the peaks appeared at 5.24 (2, 4) ppm were representing the —CH groups of HV and HB.

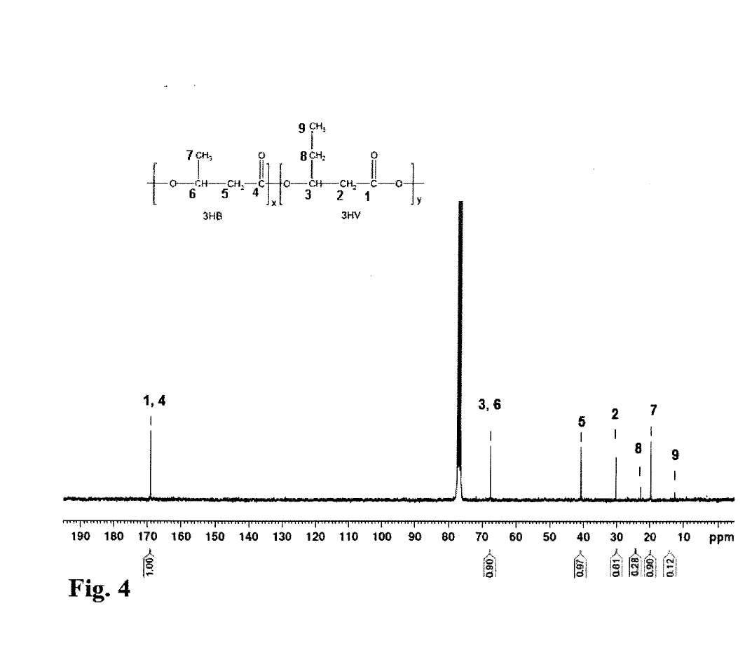

The peaks identified at 169.12, 67.60, 40.78, 30.01, 23.58, 19.75 and 9.39 ppm in the ¹³C-NMR spectrum were corresponding to the C═O, —CH (HB & HV side groups), —CH₂ (HB side group), —CH₂ (HV side group), —CH₃ (HB side group) and —CH₃ (HV side group), respectively. The peak appeared at 77 ppm was attributed to CdCl₃ (FIG. 4).

### Example 2

### Preparation and Characterization of Nanoparticles

About 40 mg of scl-PHA and 1 mg of EPT (99.98%) was dissolved in 2 mL dichloromethane by stirring at room temperature. About 20 mL of 0.9% (w/v) polyvinyl alcohol (Mw=2.2×10⁴, 88% hydrolyzed) solution was sonicated for 3 min and added into organic solution. Then, ultrasonication was continued for 15 min. The mixture was gently stirred at room temperature for 8 h. The nanoparticles were collected by centrifugation at 1.9×10⁴ g for 45 min, followed by the double wash with Milli-Q water. The blank PHBV nanoparticles were prepared by the same method without taking the EPT in organic solution. The particle size and zeta potential of the nanoparticles diluted in Milli-Q water at concentration 1 mg/mL was measured by dynamic light scattering machine Zetasizer Nano ZS (Malvern Instruments, Worcestershire, U.K.). The surface morphology of blank and EPT loaded nanoparticles was evaluated by SEM. A drop of the nanoparticles suspension was placed on a piece of cover glass and left for air drying at room temperature. The cover glass containing dried nanoparticles suspension was mounted on aluminum stumps and coated with gold in JFC1500 ion sputtering device for 1.5 min at 290° A and then examined under analytical SEM (JSM-6490A, JEOL, Japan).

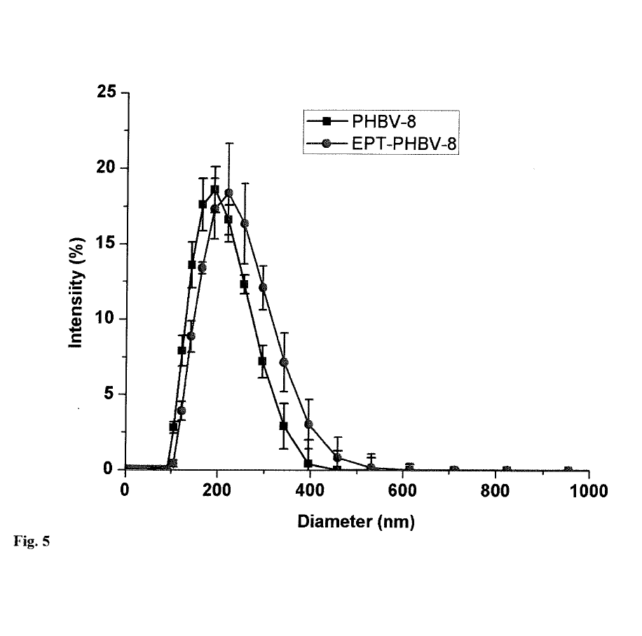

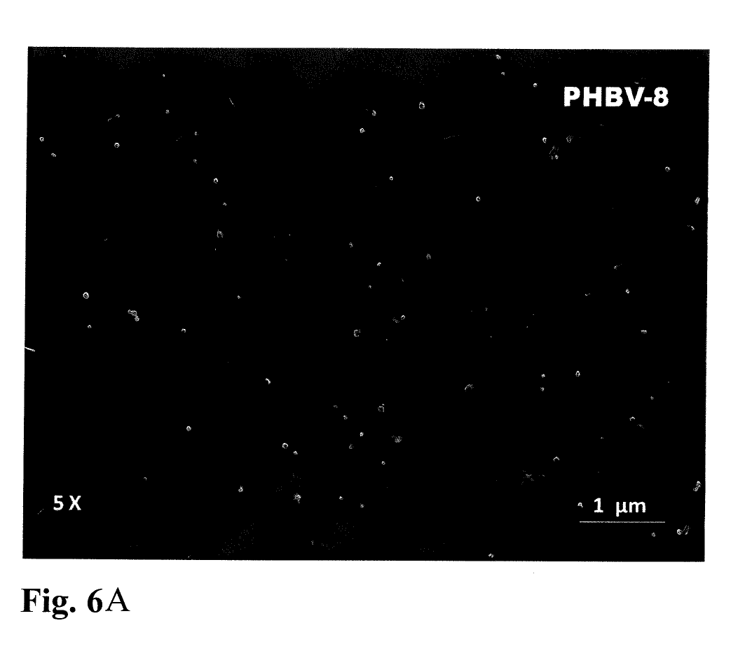

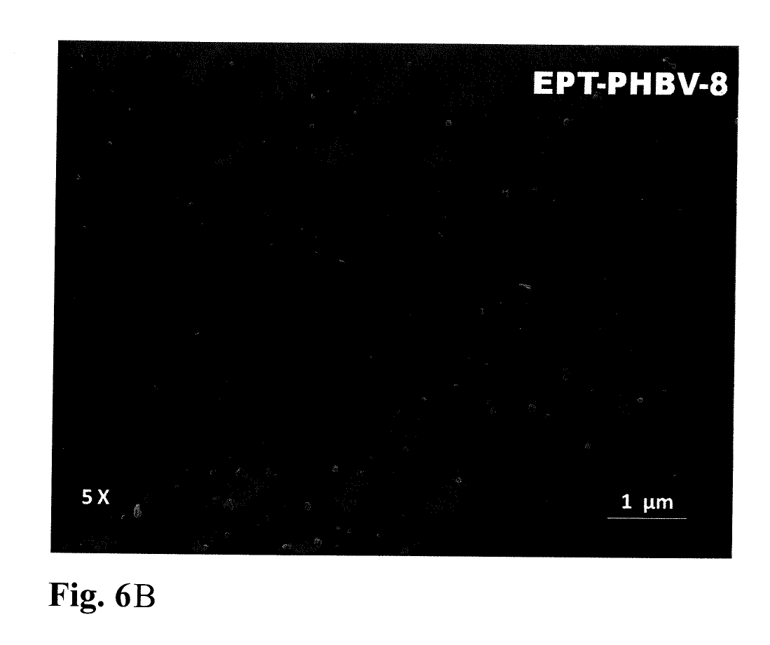

The particle size for blank and EPT loaded scl-PHA nanoparticles and their distributions were shown in FIG. 5. The average size of blank and drug loaded scl-PHA nanoparticles was 190 to 220 nm with a polydispersity index of 0.12 to 0.22. The zeta potential values of blank and EPT loaded PHA nanoparticles was −15.41 and −16.78 (Table 1). The scanning electron micrographs of blank and drug loaded scl-PHA nanoparticles revealed the regular smooth spherical shape with a slight increase in nanoparticles size after EPT loading (FIG. 6). The diameters of EPT loaded PHA nanoparticles determined by SEM were in close agreement to size determined by DLS.

TABLE 1
The sizes, polydispersity index and zeta potential values
of blank and drug loaded scl-PHA nanoparticles.

| | | | Zeta |
|---|---|---|---|
| Nanoparticles | Diameter (nm) | Polydispersity Index | potential (mV) |
| PHBV | 190.07 ± 1.08 | 0.12 ± 0.05 | −15.41 ± 0.87 |
| EPT-PHBV | 220.45 ± 2.15 | 0.22 ± 0.75 | −16.78 ± 0.53 |

### Example 3

### Drug Encapsulation Efficiency

The amount of EPT loaded in scl-PHA nanoparticles was determined by EPT UV-absorption. About 2 mg EPT loaded nanoparticles were diluted 100 times in DMSO (Sigma) and vortexed to release the drug into DMSO. The absorbance of 80 μL of the solution was then determined on a UV-Vis spectrophotometer (Biochrom Ultraspec 4300 Pro, Cambridge, England) using a quartz microcell with a 1 cm light path. The standard curve for EPT in DMSO was prepared over the concentration range from (3-20 μM) from the 5 mM stock solution of EPT. The drug loading efficiency was calculated by using the following equation;

$$Drug encapsulation efficency ( \% ) = \frac{Concentration of drug loaded in nanopaticles}{Concentration of drug added} \times 100$$

### Results

The drug encapsulation efficiency of scl-PHA nanoparticles for EPT was found to be 50.43±4.85%.

### Example 4

### In Vitro Cytotoxicity

The cellular toxicity of blank and EPT loaded scl-PHA nanoparticles as prepared in Example 2 were determined by using cancer cells line A549. The cells were cultured in Dulbecco's Modified Eagle's Medium (DMEM) containing 1% (v/v) Penicillin-Streptomycin (P/S, 10,000 U) antibiotic and 10% (v/v) Fetal Bovine Serum (FBS) and incubated for 24 h at 37° C. and 5% CO₂. When cells grew to reach 95% confluence, they were detached from the cell culture flasks using trypsin-EDTA. The cell suspension was centrifuged at 900 rpm for 5 min and then resuspended in culture medium to get the final concentration of 1×10⁵ cells/mL. About 200 μL of the cell suspensions were transferred to each well of a clear, flat bottom 96-well plate (Costar) and incubated for 24 h. On treatment day, medium was removed and fresh medium containing 4-fold serial dilutions i.e., 250.0 μg/mL (4 times), 62.5 μg/mL (8 times) and 1.6 μg/mL (12 times) of blank and EPT loaded scl-PHA nanoparticles was added. The toxicity of the nanoparticles was checked after 24, 48 and 72 h of treatment using 3-(4,5-dimethylthiazole-2-yl)-2,5-diphenyl tetrazolium (MTT) viability assay. About 5 mg of MTT was dissolved in 1 mL physiological buffer saline (PBS) solution, followed by 10 times dilution in culture medium. About 100 μL of MTT solution were added to each well after the removal of treatments and incubated for 4 h before the addition of 100 μl, of the solubilization solution (anhydrous isopropanol containing 0.1 N HCl and 10% Triton X-100). The plates were then incubated overnight and absorbance was taken at 570 nm on a microplate reader (BMG FLUOstar OPTIMA) and subtracted from the background signals at 690 nm. The cell % inhibition was calculated relative to wells containing only cell cultures without nanoparticles by using the following formula:

$$Cell \% inhibition = \frac{A [ test ]}{A [ control ]} \times 100$$

Table 2 showed the % inhibition of cancer cell line A549 for two different formulations (blank and drug loaded) of scl-PHA nanoparticles at specific time intervals as determined by MTT assay. After 72 h of incubation the % inhibition of cancer cells line A549 for EPT scl-PHA nanoparticles at 250 μg/mL concentration was ≦72.01% approximately more than two fold (32.38%) higher in comparison to free EPT which indicated the improve drug release to its target site. The inner core of scl-PHA nanoparticles has provided efficient solubilization sites for EPT and its slower degradation facilitate the release of drug from nanoparticles in a sustained manner for a prolonged period of time by the surface erosion. Then EPT entered into the cell cytoplasm due to its hydrophobic characteristic, small size and showed antitumor activity by intercalating with DNA [Singh et al. (1994) Biochemistry 33; 10271-85], inhibiting topoisomerase II activity [Shi et al. (2010) Nano Lett 10; 3223-3230; Gleeson (2008) J Med Chem 51; 817-834; Froelich-Ammon et al. (1995) J Biol Chem 270; 14998-5004] and formation of covalent DNA adducts which was mediated by cytochrome P450 and peroxidases [Poljaková et al. (2009) Collect Czechoslov Chem Commun 71:1169-85].

TABLE 2
In vitro cytotoxicity tests of blank and EPT loaded scl-PHA nanoparticles at
different concentrations for A549 cells. The bars represent the average +/− SD

| | Relative % inhibition of scl-PHA nanoparticles for A549 cells | | | | | | | |
|---|---|---|---|---|---|---|---|---|
| | Drug loaded nanoparticles | | | | | Blank nanoparticles | | |
| Time | 250 | 62.5 | 15.6 | | | 250 | 62.5 | 15.6 |
| (h) | (μg/mL) | (μg/mL) | (μg/mL) | EPT | Water | (μg/mL) | (μg/mL) | (μg/mL) |
| 24 | 25.65 ± 1.43 | 19.71 ± 1.45 | 12.89 ± 0.72 | 10.02 ± 0.86 | 10.46 ± 1.49 | 6.42 ± 1.69 | 4.41 ± 1.52 | 3.07 ± 1.72 |
| 48 | 56.50 ± 1.67 | 40.96 ± 1.30 | 35.77 ± 1.49 | 25.40 ± 1.35 | 15.91 ± 0.92 | 12.76 ± 0.45 | 9.39 ± 1.46 | 7.18 ± 1.86 |
| 72 | 72.01 ± 0.68 | 62.95 ± 1.04 | 55.99 ± 1.28 | 32.38 ± 0.95 | 20.31 ± 1.11 | 17.19 ± 1.21 | 15.34 ± 0.89 | 13.02 ± 0.97 |

Results were expressed as mean ± S.D. of three independent experiments

What is claimed is:

1. An oral drug delivery system comprising an anticancer drug ellipticine encapsulated in a nanoparticle matrix of poly-3-hydroxybutyrate-co-8 mol % 3-hydroxyvalerate (PHBV) for use in cancer patients in need for treatment.

2. The oral drug delivery system of claim 1, wherein the matrix is loaded with at least 50% with ellipticine.

3. The oral drug delivery system of claim 1, wherein the PHBV is obtained by a fermentation process.

4. The oral drug delivery system according to claim 1, wherein the PHBV is composed of 92 mol % of 3-HB and 8 mol % of 3-HV.

5. The oral drug delivery system according to claim 1, wherein viscosity average molecular weight of the PHBV is 15.70 kDa using chloroform as a solvent to test the viscosity.

6. The oral drug delivery system according to claim 1, wherein the average size of PHBV nanoparticles is 190 to 220 nm.

7. The oral drug delivery system according to claim 1, wherein the PHBV nanoparticles have a polydispersity index of 0.12 to 0.22.
